(19) United States
(12) Patent Application Publication
(10) Pub. No.: US 2015/0159215 A1
(43) Pub. Date: Jun. 11, 2015

(54) Method for Determining Nutritional Requirements of Industrial Animal

(71) Applicant: AJINOMOTO CO., INC., Tokyo (JP)

(72) Inventor: Tetsuya Takimoto, Kanagawa (JP)

(73) Assignee: AJINOMOTO CO., INC., Tokyo (JP)

(21) Appl. No.: 14/627,251

(22) Filed: Feb. 20, 2015

Related U.S. Application Data
(63) Continuation of application No. PCT/JP2013/072317, filed on Aug. 21, 2013.

(30) Foreign Application Priority Data
Aug. 21, 2012 (JP) 2012-182206

(51) Int. Cl. C12Q 1/68; A23K 1/16

(52) U.S. Cl. CPC C12Q 1/6876; A23K 1/16; C12Q 2600/158; C12Q 2600/16

(57) ABSTRACT

A means for determining the nutritional requirements of an industrial animal is provided. The objects can be achieved by identifying an mRNA the expression of which changes before and/or after a change in physiological condition in an industrial animal by comparing mRNA expression data obtained before and after the change in physiological condition, identifying a metabolic pathway of which the expression changes before and after the change in the physiological condition on the basis of the identified mRNA, and identifying a metabolite that is highly required by the industrial animal before or after the change in physiological condition on the basis of the identified metabolic pathway.

This application is a Continuation of, and claims priority under 35 U.S.C. §120 to, International Application No. PCT/JP2013/072317, filed Aug. 21, 2013, and claims priority therethrough under 35 U.S.C. §119 to Japanese Patent Application No. 2012-182206, filed Aug. 21, 2012, the entireties of which are incorporated by reference herein.

### BACKGROUND OF THE INVENTION

1. Field of the Invention

The present invention relates to a method for determining the nutritional requirements of an industrial animal, as well as a feed composition for an industrial animal and methods of producing such a feed composition.

2. Brief Description of the Related Art

In the breeding of industrial animals such as mammals, birds, fish, and crustaceans, the development of feed that is able to significantly promote growth is highly desirable. For example, regarding fish, mixed feeds that are currently widely used have been developed based on the natural feeds of fish, particularly small fish. These mixed feeds are produced by, for example, granulating raw materials such as fish meal, fats and oils, cereals, chaff and bran, vitamins, and minerals using a pellet machine or an extruder.

Since it is considered that the nutritional requirements of an industrial animal will differ depending on the type of the animal and the growth stage of the animal, it is desirable to develop mixed feeds according to these parameters for use in breeding of the industrial animal.

For example, recent literature has presented the possibility of predicting the nutritional requirements of humans based on genome information (Tamura T, et al., Genome Inform., 2010 January, 22:176-90 and Romero P, et al., Genome Biol., 2005;6(1):R2. Epub 2004 Dec 22). Moreover, in the pet industry, a method has been reported for providing a nutritional prescription suitable for an objective pathological condition by obtaining mRNAs from a normal model animal and a corresponding animal having the pathological condition, comparing the expression amounts of the mRNAs, and collating the results with known functional nutrient information (Japanese Patent Laid-open (Kohyo) No. 2010-502198).

However, a method for predicting unknown nutritional requirements specific to the growth stage of an industrial animal has not been previously reported.

### SUMMARY OF THE INVENTION

An aspect of the present invention is to provide a means for determining the nutritional requirements of an industrial animal.

Transcriptome analysis was performed for change in the expression of mRNA before and after a change in the food environment in Fundulus heteroclitus (mummichog) and mouse, a change in the expression of metabolic pathways was identified on the basis of the above analysis, and thereby metabolites more highly required before or after the change in the food environment were identified.

It is as aspect of the present invention to provide a method for identifying a metabolite that is highly required by an industrial animal, which comprises: identifying an mRNA, the expression of which changes before and after a change in physiological condition of the industrial animal, comprising comparing expression data of said mRNA obtained before and after the change in physiological condition; identifying a metabolic pathway of which the expression changes before and after the change in the physiological condition on the basis of the identified mRNA; and identifying a metabolite that is highly required by the industrial animal before or after the change in physiological condition on the basis of the identified metabolic pathway.

It is a further aspect of the present invention to provide a method as described above, which comprises obtaining mRNA expression data.

It is a further aspect of the present invention to provide a method as described above, wherein when the expression of a certain metabolic pathway increases before or after the change in the physiological condition, a metabolite corresponding to upstream of the metabolic pathway and/or a metabolite corresponding to downstream of the metabolic pathway is identified as a metabolite highly required by the industrial animal before or after the change in the physiological condition.

It is a further aspect of the present invention to provide a method as described above, wherein when the expression of a certain metabolic pathway decreases before or after the change in the physiological condition, a metabolite corresponding to downstream of the metabolic pathway and important for growth or life activity, and/or a metabolite corresponding to upstream of the metabolic pathway is identified as a metabolite highly required by the industrial animal before or after the change in the physiological condition.

It is a further aspect of the present invention to provide a method as described above, wherein said change in physiological condition is selected from the group consisting of ontogenesis, a transition in a baby animal from the fetal period to the period after birth, a transition in a mother animal from the gestation period to the non-gestation period, start of feeding of a baby animal from a source other than the mother, a transition in a mother animal from a non-lactation period and a lactation period, and a transition in a baby animal from a lactation period to a non-lactation period.

It is an aspect of the present invention to provide a method for producing a feed composition, which comprises: identifying a metabolite that is highly required by an industrial animal before or after a change in physiological condition by the method as described above; and adding the metabolite to a composition.

### DESCRIPTION OF THE PREFERRED EMBODIMENTS

### <1> Method of the Present Invention

The method as described herein is a method for determining the nutritional requirements of an industrial animal. Specifically, it is a method for identifying metabolites highly required by industrial animals. Feed formulations and compositions are also described which contain such required metabolites.

The method as described herein includes the following steps: (1) identifying an mRNA the expression of which changes before and after a change in a physiological condition of the industrial animal by comparing mRNA expression data obtained before and after the change in physiological condition , (2) identifying a metabolic pathway of which the expression changes before and after the change in the physiological condition on the basis of the identified mRNA and (3) identifying a metabolite highly required by the industrial animal either before or after the change in physiological condition before or after the change in the physiological condition on the basis of the identified metabolic pathway.

The term “industrial animal” can include domesticated animals and fish.

The term “domesticated animals” or “domestic animals” can include, for example, animals farmed for production of various products, such as milk, meat, eggs, hair, leather, or their labor, as well as animals raised for companionship and pets, such as dogs and cats. Specific examples of the “domestic animals” can include, for example, cow, pig, chicken, horse, turkey, sheep, goat, dog, and cat. Among these, pig and chicken are particular examples. As the chicken, chickens raised for their meat and/or for production of eggs are other particular examples, and broilers are another particular example.

The phrase “fish that are farmed” can include fish and crustaceans. Specific examples of fish can include, for example, tuna, bonito, yellowtail, greater amberjack, amberjack, sea bream, salmon, cod, trout, rainbow trout, flatfish, tiger globefish, filefish, horse mackerel, grouper, eel, carp, catfish, tilapia, barramundi, grass carp, silver carp, crucian carp, and Fundulus heteroclitus, also known as mummichog. Specific examples of the crustaceans can include, for example, tiger shrimp black tiger shrimp, vannamei shrimp, and crab.

Examples of the “change in a physiological condition” can include a change in the food environment, such as the type or source of food, or the manner of feeding. Examples of the change in the food environment include, for example, ontogenesis, the transition in a baby animal from the fetal period to the period after birth, the transition in a mother animal from the gestation period to the non-gestation period or the reverse, the start of feeding of a baby animal from a source other than the mother, the transition in a mother animal between lactation and non-lactation periods or the reverse, and the transition in a baby animal between the lactation and non-lactation periods, also called ablactation or weaning.

Any mRNA expression data opened to public may be used. Examples of mRNA expression data opened to public can include, for example, data registered in public databases and data disclosed in published literature. Specifically, for example, the data registered in the gene expression database GEO of NCBI (www.ncbi.nlm nih gov/gds) can be used.

Alternatively, mRNA expression data can be obtained by conducting gene expression analysis. That is, the method as described herein may use mRNA expression data that is obtained before and after the change in physiological condition of an industrial animal. The gene expression analysis can be conducted by, for example, a known method. Specifically, mRNA expression data before and after the change in physiological condition in an industrial animal can be obtained by, for example, extracting total RNA from the industrial animal before and after the change in physiological condition, and performing transcriptome analysis.

The mRNA expression data may be extracted from the entire industrial animal, or from a targeted tissue of the industrial animal. Examples of the targeted tissue can include, for example, the gastrointestinal tract. Examples of tissues of the gastrointestinal tract can include, for example, the ileum.

The mRNA expression data may be the total mRNA, or may be a part of the mRNA. Examples of a part of mRNA can include, for example, mRNA corresponding to or involved in a specific metabolic pathway. Examples of the specific metabolic pathway can include, for example, a metabolic pathway relevant to a specific metabolite. Examples of the “metabolic pathway relevant to a specific metabolite” can include a metabolic pathway that generates the specific metabolite, and a metabolic pathway that metabolizes the specific metabolite into another metabolite. Specific examples of the specific metabolite can include, for example, saccharides, amino acids, lipids, and vitamins. The “metabolic pathway” may consist of a single reaction step, two or more sequential reaction steps, or a combination thereof. The “mRNA involved in a metabolic pathway” can refer to one or more mRNAs that are translated into one or more kinds of proteins that catalyze one or more reaction steps of the metabolic pathway.

In the method described herein, an mRNA, the expression level of which changes, before and after a change in physiological condition of an industrial animal is identified by comparing mRNA expression data obtained before and after the change in physiological condition. Such mRNA, the expression of which changes before and after a change in the physiological condition can also be referred to as “expression-changing mRNA”. The degree of change in the expression is not particularly limited so long as it is a statistically significant change. To conduct the statistical analysis, a statistical analysis tool can be used. Specific examples of statistical analysis tools can include, for example, the open source statistical analysis software “R” (www.r-project.org/), and the web tool GEO2R (www.ncbi.nlm nih gov/geo/geo2r/). The comparison may be performed for the total mRNA or for a part of the mRNA.

Examples of a part of mRNA can include, for example, mRNA corresponding to a specific metabolic pathway.

In the method described herein, a metabolic pathway of which the expression changes before and after the change in the physiological condition is identified on the basis of the expression-changing mRNA identified above. Specifically, the metabolic pathway corresponding to the expression-changing mRNA is identified as the metabolic pathway of which the expression changes before and after the change in the physiological condition. The “metabolic pathway corresponding to the expression-changing mRNA” refers to a metabolic pathway that is catalyzed by a protein translated from the mRNA. The “metabolic pathway corresponding to the expression-changing mRNA” can refer to a metabolic pathway that is catalyzed by a protein translated from the mRNA. The metabolic pathway corresponding to the expression-changing mRNA can be identified on the basis of the function of the protein translated from the mRNA.

The function of a protein can be estimated by comparing the amino acid sequence of the protein with the amino acid sequence(s) of known protein(s). The method as described herein may include estimating the function of a protein, such as mentioned above. The “known protein” is not particularly limited so long as it is a protein or a functional motif of which the amino acid sequence and function are known. The amino acid sequence of a known protein can be an experimentally determined amino acid sequence, or can be an amino acid sequence estimated from genome information or from a gene sequence. The function of a known protein can be an experimentally determined function, or can be a function estimated by comparison with the amino acid sequence(s) of other known protein(s). The function of a protein can be estimated by, for example, comparing the amino acid sequence of the protein with the amino acid sequence(s) of known protein(s) registered at a publicly known database. Amino acid sequences can be compared by, for example, a homology search, motif prediction, or the like. A homology search can be performed by using, for example, BLAST.

The amino acid sequence of a protein can be obtained from, for example, a publicly known database. Although such a database is not particularly limited, examples thereof include, for example, NCBI (www.ncbi.nlm nih gov/), Ensembl (asia.ensembl.org/index.html), DDBJ (DNA Data Bank of Japan, www.ddbj.nig.ac.jp/), and EMBL (European Molecular Biology Laboratory, www.embl.org/). Specifically, for example, on the ftp site of Ensembl (asia.ensembl.org/info/data/ftp/index.html), by selecting “Protein sequence (FASTA)” of an objective biological species, the amino acid sequences of all the proteins of that biological species predicted from the genome information can be downloaded in the FASTA format. Amino acid sequence(s) used in the method of the present invention can also be referred to as “amino acid sequence data”.

Amino acid sequence data of a protein can consist of amino acid sequence data deduced from genome information (nucleotide sequence), or can contain other amino acid sequence data. For example, amino acid sequence data of a protein can contain experimentally determined amino acid sequence data.

It is sufficient that amino acid sequence data of a protein is described in a format usable for estimating the function of the protein. The “format usable for estimating the function of a protein” can be appropriately chosen depending on software or an algorithm thereof used for estimating the function of the protein. For example, the amino acid sequence of a protein can be described in the FASTA format. The FASTA format is a format containing one or more pieces of sequence data (for example, amino acid sequence data of protein(s)) divided by header line(s). FASTA format including two or more pieces of sequence data is also called multi-FASTA format. The multi-FASTA format is especially useful for annotating two or more kinds of proteins at once. Specifically, for example, when the annotation is performed with KAAS described later, amino acid sequences described in the multi-FASTA format can be batch-processed.

Specifically, for example, amino acid sequence data of a protein described in the FASTA format can be processed on KAAS (KEGG automatic annotation server) of KEGG (Kyoto Encyclopedia of Genes and Genomes, www.genome.jp/kegg/) or of iKeg, which is a local server of KEGG, and annotated on the basis of KO (KEGG Orthology), so as to estimate the function of the protein. In such a case, annotation of an objective protein is performed by comparison with the amino acid sequence(s) of known protein(s) to which annotation based on KO on the KEGG database has been added. As for the details of KEGG, for example, Kanehisa M, et al., Nucleic Acids Res., 34, D354-357 (2006), or Kanehisa M, et al., Nucleic Acids Res., 36, D480-D484 (2008) can be referred to. As for the details of KAAS, for example, Moriya Y, et al., Nucleic Acids Res., 35 (Web Server issue):W182-5, Jul. 2007 can be referred to. Processing of amino acid sequence data can be performed for each protein individually, or for two or more kinds of proteins at once. For example, by processing data including the amino acid sequences of two or more kinds of proteins described in the multi-FASTA format on KAAS, annotation of the two or more kinds of proteins based on KO can be performed at once. Either the function of a fish protein or the function of a protein of a reference organism can be estimated separately, or they can be estimated simultaneously.

In addition, the method for estimating the function of a protein is not limited to the annotation based on KO. The function of a protein can be estimated by any method that enables functional classification of a protein based on amino acid sequence. For example, the function of a protein can be estimated by comparison (for example, a homology search) with the amino acid sequence(s) of already annotated protein(s) of Swiss-Prot or TrEMBL in UniProtKB (UniProt Knowledgebase, www.uniprot.org/help/uniprotkb).

In addition, “estimating the function of a protein by comparing the amino acid sequence of the protein with the amino acid sequence(s) of known protein(s)” includes estimation by the processing of arbitrary data that can be converted into the amino acid sequence of the protein, as well as estimation by direct processing of amino acid sequence data of the protein. That is, the function of a protein can be estimated on the basis of arbitrary data that can be converted into the amino acid sequence of the protein. Examples of such data include, for example, the nucleotide sequence of a gene. Such data can be converted into the amino acid sequence of a protein beforehand or at the time of estimation of the function (at the time of annotation), and then can be used.

A metabolic pathway corresponding to mRNA the expression level of which changes before and after a change in a physiological status may be identified for all the expression-changing mRNAs, or only some of the expression-changing mRNAs. For example, such a metabolic pathway may be identified for a certain number of mRNAs showing the greatest degrees of the expression change. Although this “certain number of mRNAs” is not particularly limited, it may be, for example, 400, 200, or 100 mRNAs.

Specifically, a metabolic pathway corresponding to mRNA the expression level of which changes before and after a change in a physiological condition can be identified by, for example, the PAGE (Parametric Analysis of Gene Set Enrichment) method (Kim and Volsky, BMC Bioinformatics, 2005, 6:144).

The method as described herein may include a step of mapping the functions of proteins translated from the expression-changing mRNAs to a metabolic pathway. By mapping the functions of proteins translated from expression-changing mRNAs to a metabolic pathway, the objective metabolic pathway can be visualized. Such mapping can be performed by using, for example, the KEGG mapper (www.genome.jp/kegg/mapper.html).

Based on the above-identified metabolic pathway corresponding to mRNA the expression level of which changes before and after a change in a physiological condition, metabolite(s) highly required by an industrial animal before or after the change in the physiological condition can be identified. This “highly required” means that the metabolite is required in different amounts either before or after the change in the physiological condition. Specifically, a larger amount of the metabolite is required after the change in the physiological condition as compared to before the change in the physiological condition, or a larger amount of the metabolite is required before the change in the physiological condition as compared to after the change in the physiological condition.

In the method as described herein, when the expression of a certain metabolic pathway increases before or after a change in a physiological condition, a metabolite produced in the upstream portion of the metabolic pathway and/or a metabolite produced in the downstream portion of the metabolic pathway may be determined to be a metabolite that is highly required by the industrial animal before or after the change in the physiological condition. The phrase that “the expression of a metabolic pathway increases before a change in physiological condition” means that the degree of the expression of the metabolic pathway before the change in physiological condition is higher than that observed after the change in physiological condition. The phrase that “the expression of a metabolic pathway increases after the change in physiological condition” means that the degree of the expression of the metabolic pathway after the change in physiological condition is higher than that observed before the change in physiological condition.

In the method as described herein, when the expression of the mRNA corresponding to a certain metabolic pathway decreases before or after the change in a physiological condition, a metabolite produced in the upstream portion of the metabolic pathway may be determined to be a metabolite that is highly required by the industrial animal before or after the change in physiological condition. Furthermore, in the method described herein, when the expression of the mRNA corresponding to a certain metabolic pathway decreases before or after a change in physiological condition, a metabolite produced in the downstream portion of the metabolic pathway and important for growth or life activity may be identified as being highly required by the industrial animal before or after the change in physiological condition. Examples of metabolites important for growth or life activity include, for example, glucose, aliphatic acids, and amino acids. The phrase that “the expression of a metabolic pathway decreases before the change in physiological condition” means that the degree of the expression before the change in physiological condition is lower than that observed after the change in physiological condition. The phrase that “the expression of a metabolic pathway decreases after change in physiological condition” means that the degree of the expression after the change in physiological condition is lower than that observed before the change in physiological condition.

Examples of “metabolite produced in the upstream portion of a metabolic pathway” can include a metabolite that serves as the starting substance of the metabolic pathway, and a metabolite that is metabolized to then generate such a starting substance. Although the portion of a metabolic pathway that is considered upstream can vary depending on the number of reactions in the metabolic pathway and the presence of bypass pathway(s), it may be up to, for example, the 20th metabolite, 10th metabolite, 5th metabolite, or third metabolite, on the upstream side counting the starting substance of the metabolic pathway as the first metabolite.

Examples of a “metabolite produced in the downstream portion of a metabolic pathway” can include the direct product of the metabolic pathway, as well as further metabolized products of the direct product. Although the portion of a pathway that is considered downstream can vary depending on the number of reactions in the metabolic pathway and the presence of bypass pathway(s), it may be up to, for example, the 20th metabolite, 10th metabolite, 5th metabolite, or third metabolite on the downstream side, counting the direct product of the metabolic pathway as the first metabolite.

Furthermore, a “metabolic pathway” can include an uptake system for a metabolite. The uptake system for a metabolite may be one that changes the metabolite, or may be one that does not change the metabolite. Examples of the “metabolite produced in the upstream portion of a metabolic pathway” in relation to an uptake system for a metabolite can include a metabolite that is to be imported by the uptake system. Examples of the “metabolite produced in the downstream portion of a metabolic pathway” in relation to an uptake system for a metabolite include a metabolite that has been imported by the uptake system and a metabolite that is produced from the imported metabolite by further metabolism.

Whether the nutritional requirements of an industrial animal have been correctly determined by the method as described herein can be confirmed by providing to the an industrial animal feed produced according to the determined nutritional requirements, for example, feed containing the metabolite(s) determined to be highly required by the industrial animal, and to another industrial animal, control feed, for example, feed not containing the metabolite(s), and comparing the degree of growth obtained .

### <2> Program of the Present Invention

The program as described herein is a program for making a computer execute the steps of the method as described herein.

That is, a program is described for making a computer execute the following steps (1) to (3):

(1) a step of identifying an mRNA the expression of which changes before and after a change in physiological condition in an industrial animal by comparing mRNA expression data obtained before and after the change in physiological condition,

(2) a step of identifying a metabolic pathway of which the expression changes before and after the change in physiological condition on the basis of the identified mRNA, and

(3) a step of identifying a metabolite that is highly required by the industrial animal before or after the change in physiological condition on the basis of the identified metabolic pathway.

The program of the present invention may make a computer execute a step of estimating the function of a protein in the industrial animal by comparing the amino acid sequence of the protein of the industrial animal with the amino acid sequence of a known protein.

The program of the present invention can be recorded on a recording medium readable by a computer, and provided. A “recording medium readable by a computer” recited herein refers to a recording medium on which information such as data and program can be stored by electric action, magnetic action, optical action, mechanical action, chemical action, or the like, and from which the stored information can be read by a computer. Examples of such a recording medium include, for example, floppy disc (registered trademark), magnetic optical disc, CD-ROM, CD-R/W, DVD-ROM, DVD-R/W, DVD-RAM, DAT, 8 mm tape, memory card, hard disk, ROM (read only memory), SSD, and so forth. As for the program of the present invention, the respective steps to be executed by a computer can be recorded as a single program collectively, or can be recorded as separate programs individually or as an arbitrary combination of separate programs.

<3> Feed Composition of the Present Invention

A feed composition can be produced by adding the metabolite determined to be highly required by an industrial animal by the method as described herein to a raw material. That is, the feed composition as described herein can contain one or more metabolites that are highly required by an industrial animal before or after a change of physiological condition.

The feed composition as described herein may contain only the highly required metabolite identified above, or may further contain other component(s).

The “other component(s)” is/are not particularly limited, so long as it is a substance that can be orally ingested by the industrial animal, and for example, can be ingredients typically blended and used in feed or drugs. That is, the feed composition can be produced in the same manner by using the same raw materials as those used for usual feeds for feeding an industrial animal, except that the highly required metabolite identified above is added to the raw materials and/or other components.

For example, unless the effect of the method as described herein is degraded, one or more raw materials can be blended into the feed composition as described herein. Examples of the feed raw materials include, for example, bran such as wheat bran, rice bran, barley bran, and millet bran; production lees such as tofu lees, starch lees, copra meal, sake lees, soy sauce lees, brewer's lees, shochu lees, and fruit or vegetable juice lees; cereals such as corn, rice, wheat, barley, and oat; oil meals such as soybean meal, rapeseed meal, cottonseed meal, linseed meal, sesame meal, and sunflower seed meal; animal material feeds such as fish meal, casein, skim milk powder, dried whey, meat bone meal, meat meal, feather meal, and powdered blood; and leaf meals such as alfalfa meal.

Furthermore, for example, unless the effect of the method described herein is degraded, one or more kinds of components selected from excipients, fillers, nutrition reinforcers, feed additives, and so forth can also be blended into the feed composition as described herein. Examples of the excipients include, for example, cellulose derivatives such as carboxymethylcellulose. Examples of the fillers include, for example, dextrin and starch. Examples of the nutrition reinforcers include, for example, vitamins and minerals. Examples of the feed additives include, for example, enzyme preparations and live cell preparations.

The form of the feed composition is not particularly limited, unless the effect of the method described herein is degraded. The feed composition may be in any form, such as powder, granule, liquid, paste, and cube.

The feed composition can be used in the breeding of an industrial animal.

The feed composition may be administered by itself to an industrial animal, or may be administered in combination with other feed. When the feed composition is administered in combination with other feed, the feed composition as described herein and the other feed may be administered simultaneously, or may be administered separately. When the feed composition and the other feed are administered simultaneously, for example, the feed composition can be added to the other feed, and the resulting mixture can be administered.

The feed composition may be administered once a day, or two or more times a day as divided portions. The feed composition may also be administered once per several days. The dose of the feed composition may or may not be fixed for such administrations, in terms of the amount of the required metabolite identified above.

The time of the administration of the feed composition is not particularly limited so long as it is administered before or after the change in physiological condition of an industrial animal. That is, when the feed composition contains a metabolite that is required by an industrial animal before the change in physiological condition, it may be administered to the industrial animal before the change in physiological condition. When the feed composition contains a metabolite required by an industrial animal after the change in physiological condition, it may be administered to the industrial animal after the change in physiological condition. The feed composition may be continuously administered over the entire period before or after the change in physiological condition in an industrial animal, or may be administered during only a portion of the period before or after the change in physiological condition in an industrial animal. For example, the feed composition may be continuously administered until immediately before change in physiological condition in an industrial animal, or may be continuously administered from immediately after the change in physiological condition in an industrial animal. Furthermore, continuation and discontinuation of the administration of the feed composition may be repeated during an arbitrary period before or after the change in physiological condition of an industrial animal. Furthermore, the feed composition may be administered only either before or after the change in physiological condition of an industrial animal, or may be administered both before and after the change in physiological condition of an industrial animal.

The feed composition may include, for example, feed such as mother's milk, milk substitute, pre-initial feed (pre-starter feed), initial feed (starter feed), and fattening term feed. The feed composition may also be used, for example, in combination with any feed such as mother's milk, milk substitute, pre-initial feed (pre-starter feed), initial feed (starter feed), and fattening term feed.

### EXAMPLES

The present invention will be more specifically explained with reference to the following non-limiting examples.

### Example 1

### Prediction Of Nutritional Requirements of Fundulus Heteroclitus (Mummichog) After Start Of Feeding Behavior

In this example, genes the expression of which changed before and after the start of a feeding behavior were detected in mummichog. Then, the metabolic pathways in which the genes participate were identified, and from this information, candidate nutrients that are required due to the start of a feeding behavior were determined The procedure and results are shown below.

The mRNA expression data set GSE21372, which is registered in the genetic expression database GEO (www.ncbi.nlm nih gov/gds) in NCBI and includes the results of microarray expression analysis of the processes of embryogenesis, hatching, and growth after fertilization of a mummichog egg, was obtained. The data for the period after hatching included in the obtained data set was divided into two groups, 1) the period after hatching but before the mummichog starts to feed on food found outside the egg, and 2) the period after the mummichog starts to feed on food found outside the egg. Hence, the change in physiological condition is the start of feeding for the first time on food found outside the egg after hatching. A statistical analysis of the change in the amount of mRNA expression between these two periods was conducted by using the free statistical analysis software “R” (www.r-project.org/). Enzyme numbers were extracted from the microarray platform used in the test, and assigned to the results of expression change analysis. Among the genes to which the enzyme numbers were assigned, the top 200 genes showing the highest degree of change in expression were extracted.

The enzyme numbers of the extracted genes were mapped to metabolic pathways using the KEGG mapper (www.genome.jp/kegg/mapper.html) to thereby visualize the metabolic pathways that characteristically changed to the highest degree after the start of feeding behavior.

Considering the details of the metabolic pathways of which the expression changed, candidates for nutrients highly required after the start of feeding behavior were determined Specifically, candidate nutrients were selected by, for example, a) identifying a metabolite generated via a metabolic pathway that includes mRNA the expression of which decreased, and wherein said metabolite is important for growth or life activity, b) identifying a metabolite produced in the upstream portion of a metabolic pathway that includes mRNA the expression of which increased, and/or c) identifying a metabolite produced from the metabolite identified in b) by further metabolism, and by regarding the metabolites identified in a) to c) as metabolites that must be supplied exogenously after the start of feeding. The candidate nutrients that were determined to be highly required after the start of feeding after hatching are shown in Table 1.

TABLE 1

| | | | | | | | | | gene |
|---|---|---|---|---|---|---|---|---|---|
| | | | | | | | | | expression |
| | | | | | | | | | after stating |
| | | | | | | | FCR | | the feeding |
| nutrients | | pathway | gene name | FCR | EC | GB Acce | (log2) | rank_wad | action |
| betain | ko00260 | Glycine, serine and | Betaine aldehyde dehydrogenase | 0.63 | 1.2.1.8 | CN980759 | −0.66 | 588 | − |
| | | threonine metabolism | (EC 1.2.1.8) (BADH)_1643 | | | | | | |
| | ko00260 | Glycine, serine and | Alcohol dehydrogenase | 0.76 | 1.1.1.1 | CN980373 | −0.40 | 1134 | − |
| | | threonine metabolism | (EC 1.1.1.1)_6635 | | | | | | |
| peptide | ko04974 | Protein digestion and | Carboxypeptidase B precursor | 1.47 | 3.4.17.2 | CN984239 | 0.56 | 257 | + |
| | | absorption | (EC 3.4.17.2)_6300 | | | | | | |
| protein | ko04974 | Protein digestion and | Elastase-2A precursor | 1.79 | 3.4.21.71 | EV450494 | 0.84 | 206 | + |
| | | absorption | (EC 3.4.21.71)_3121 | | | | | | |
| | ko04974 | Protein digestion and | Trypsin II precursor | 0.68 | 3.4.21.4 | EV451754 | −0.55 | 1019 | − |
| | | absorption | (EC 3.4.21.4) (Fragment)_3522 | | | | | | |
| | ko04974 | Protein digestion and | Chymotrypsin B (EC 3.4.21.1) | 1.35 | 3.4.21.1 | EV453512 | 0.43 | 1138 | + |
| | | absorption | [Contains: Chymotrypsin B chain A; | | | | | | |
| | | | Chymotrypsin B chain B]_4066 | | | | | | |
| myo- | ko00562 | Inositol phosphate | Inositol monophosphatase | 2.78 | 3.1.3.25 | CV822196 | 1.48 | 12 | + |
| inositol | | metabolism | (EC 3.1.3.25) (IMPase) (IMP) | | | | | | |
| | | | (Inositol-1(or 4)-monophosphatase) | | | | | | |
| | | | (Lithium-sensitive myo-inositol | | | | | | |
| | | | monophosphatase A1)_1862 | | | | | | |
| | ko00562 | Inositol phosphate | Phosphatidylinositol 4-kinase beta | 0.75 | 2.7.1.67 | CN983629 | −0.42 | 882 | − |
| | | metabolism | (EC 2.7.1.67) (PtdIns 4-kinase) | | | | | | |
| | | | (PI4Kbeta) (PI4K-beta)_873 | | | | | | |
| oligo (di) | ko04974 | Protein digestion and | Maltase-glucoamylase, intestinal | 1.65 | 3.2.1.20 | EV461792 | 0.72 | 699 | + |
| saccharide | | absorption | [Includes: Maltase (EC 3.2.1.20) | | | | | | |
| | | | (Alpha-glucosidase); | | | | | | |
| | | | Glucoamylase (EC 3.2.1.3) | | | | | | |
| | | | (Glucan 1,4-alpha-glucosidase)]_4715 | | | | | | |

### Example 2

### Predicting the Nutritional Requirements of a Mouse after Ablactation

Ablaction is defined as the act of weaning an offspring, or the cessation of milk production by a mother mammal. In this example, genes the expression of which changed before and after the change in physiological condition caused by ablactation or weaning were detected in mouse, and then the metabolic pathways in which the genes participate were identified. From this information, candidate nutrients that are requirement is increased due to ablactation were determined. The procedure and results are shown below.

The mRNA expression data set GDS2989, which is registered in the genetic expression database GEO (www.ncbi.nlm nih gov/gds) in NCBI and includes the results of microarray expression analysis of the mouse ileum up to the 32nd day after birth, was obtained. The data set was divided into two groups, that is, those before and after ablactation, and statistical analysis of the change in the mRNA expression observed before and after the ablactation was conducted by using the web tool GEO2R (www.ncbi.nlm nih gov/geo/geo2r/).

Amino acid sequence data of mouse proteins were obtained from BioMart of Ensembl (www.ensembl.org/biomart/martview/243057aba97fc1845ec768160454a20f). The obtained amino acid sequence data were processed on the KEGG Automatic Annotation Server (KAAS) of iKeg (local server of KEGG), annotation based on KEGG Orthology (KO) was collectively performed for the respective proteins, and assigned to the results of expression change analysis. Among the genes, those for which the expression significantly changed were extracted.

KO numbers of the extracted genes were mapped on metabolic pathways using the KEGG mapper (www.genome.jp/kegg/mapper.html) to thereby visualize the metabolic pathways that characteristically changed before and after ablactation.

By using the free statistical analysis software “R” (www.r-project.org/), PAGE analysis (Kim and Volsky, BMC Bioinformatics, 2005;6:144) was performed on the basis of GO terms and KEGG pathway names obtainable from GSEA (www.broadinstitute.org/gsea/index.jsp) to extract metabolic pathways that contain DNA or mRNA the expression of which significantly changed before and after the ablactation. The results obtained by using the GO terms are shown in Table 2, and the results obtained by using the KEGG pathway names are shown in Table 3.

TABLE 2

| rank | p_page | z_page | Geneset_name | GO_ID |
|---|---|---|---|---|
| 1 | 2.13E−09 | −5.98744 | PATTERN_RECOGNITION_RECEPTOR_ACTIVITY | GO: 0008329 |
| 2 | 5.38E−08 | −5.43834 | SOLUTE_SODIUM_SYMPORTER_ACTIVITY | GO: 0015370 |
| 3 | 2.37E−07 | −5.16752 | EPITHELIAL_CELL_DIFFERENTIATION | GO: 0030855 |
| 4 | 5.53E−07 | −5.00707 | BRUSH_BORDER | GO: 0005903 |
| 5 | 7.08E−07 | −4.95929 | IMMUNE_SYSTEM_PROCESS | GO: 0002376 |
| 6 | 7.43E−07 | 4.949718 | LYSOSOME_ORGANIZATION_AND_BIOGENESIS | GO: 0007040 |
| 7 | 8.23E−07 | 4.929809 | RECEPTOR_MEDIATED_ENDOCYTOSIS | GO: 0006898 |
| 8 | 1.21E−06 | 4.853702 | VACUOLE | GO: 0005773 |
| 9 | 1.34E−06 | −4.83392 | HYDROLASE_ACTIVITY_ACTING_ON_CARBON_NITROGEN_NOT_ | GO: 0016814 |
| | | | PEPTIDEBONDSIN_CYCLIC_AMIDINES | |
| 10 | 1.42E−06 | 4.821735 | VITAMIN_TRANSPORT | GO: 0051180 |
| 11 | 1.94E−06 | −4.75968 | CARBOXYLESTERASE_ACTIVITY | GO: 0004091 |
| 12 | 2.52E−06 | 4.706365 | LYSOSOMAL_MEMBRANE | GO: 0005765 |
| 13 | 3.61E−06 | 4.63281 | VACUOLE_ORGANIZATION_AND_BIOGENESIS | GO: 0007033 |
| 14 | 3.80E−06 | −4.6219 | MORPHOGENESIS_OF_AN_EPITHELIUM | GO: 0002009 |
| 15 | 3.97E−06 | 4.613015 | OXYGEN_AND_REACTIVE_OXYGEN_SPECIES_METABOLIC_PROCESS | GO: 0006800 |
| 16 | 5.61E−06 | 4.540605 | COFACTOR_TRANSPORT | GO: 0051181 |
| 17 | 6.34E−06 | 4.514574 | HYDROLASE_ACTIVITY_HYDROLYZING_O_GLYCOSYL_COMPOUNDS | GO: 0004553 |
| 18 | 7.72E−06 | −4.47277 | ANION_CHANNEL_ACTIVITY | GO: 0005253 |
| 19 | 7.72E−06 | −4.47277 | CHLORIDE_CHANNEL_ACTIVITY | GO: 0005254 |
| 20 | 7.85E−06 | −4.46913 | IMMUNE_RESPONSE | GO: 0006955 |
| 21 | 8.08E−06 | 4.462994 | VITAMIN_BINDING | GO: 0019842 |
| 22 | 8.76E−06 | −4.44564 | DEAMINASE_ACTIVITY | GO: 0019239 |
| 23 | 9.93E−06 | −4.41879 | PHOSPHOLIPASE_ACTIVITY | GO: 0004620 |
| 24 | 1.03E−05 | 4.411518 | VACUOLAR_MEMBRANE | GO: 0005774 |
| 25 | 1.11E−05 | 4.394252 | LYSOSOME | GO: 0005764 |
| 26 | 1.11E−05 | 4.394252 | LYTIC_VACUOLE | GO: 0000323 |
| 27 | 1.30E−05 | −4.36039 | METALLOEXOPEPTIDASE_ACTIVITY | GO: 0008235 |
| 28 | 1.31E−05 | 4.359225 | COPPER_ION_BINDING | GO: 0005507 |
| 29 | 1.43E−05 | −4.33945 | SERINE_TYPE_ENDOPEPTIDASE_ACTIVITY | GO: 0004252 |
| 30 | 1.58E−05 | −4.31674 | TRANSFERASE_ACTIVITY_TRANSFERRING_HEXOSYL_GROUPS | GO: 0016758 |
| 31 | 1.71E−05 | −4.29949 | OXIDOREDUCTASE_ACTIVITY_ACTING_ON_SULFUR_GROUP_ | GO: 0016667 |
| | | | OF_DONORS | |
| 32 | 1.98E−05 | −4.26731 | POSITIVE_REGULATION_OF_CELL_ADHESION | GO: 0045785 |
| 33 | 2.44E−05 | −4.22041 | PHOSPHOLIPASE_A2_ACTIVITY | GO: 0004623 |
| 34 | 3.11E−05 | −4.1653 | LEUKOCYTE_CHEMOTAXIS | GO: 0030595 |
| 35 | 5.26E−05 | −4.04385 | RESPONSE_TO_OTHER_ORGANISM | GO: 0051707 |
| 36 | 6.36E−05 | 3.99909 | HYDROLASE_ACTIVITY_ACTING_ON_GLYCOSYL_BONDS | GO: 0016798 |
| 37 | 6.56E−05 | −3.9916 | RESPONSE_TO_BIOTIC_STIMULUS | GO: 0009607 |
| 38 | 7.59E−05 | 3.95701 | VACUOLAR_PART | GO: 0044437 |
| 39 | 7.77E−05 | −3.95151 | POSITIVE_REGULATION_OF_TRANSLATION | GO: 0045727 |
| 40 | 8.81E−05 | −3.92127 | TRANSMEMBRANE_TRANSPORTER_ACTIVITY | GO: 0022857 |

Table 3

Considering the details of the metabolic pathways, candidate nutrients highly required after the ablactation were determined on the basis of the results of the PAGE analysis. Specifically, candidate nutrients were determined by, for example, a) identifying a metabolite produced in the downstream portion of a metabolic pathway that includes DNA the expression of which decreased, and wherein said metabolite is important for growth or life activity, b) identifying a metabolite produced in the upstream portion of a metabolic pathway that includes DNA the expression of which decreased, c) identifying a metabolite produced in the upstream or downstream portion of a metabolic pathway that includes DNA the expression of which increased, and/or d) identifying a metabolite produced from the metabolite identified in c) by further metabolism, and by regarding the metabolites identified in a) to d) as metabolites that must be supplied exogenously. The candidate nutrients that are required after the ablactation are shown in Table 4.

Table 4:

As described above, by estimating metabolic change on the basis of transcriptome information, nutritional requirements specifically observed after a change in physiological condition could be determined in fish and mammals. Furthermore, nutritional requirements specifically observed before a change in physiological condition can also be determined in a similar manner.

### INDUSTRIAL APPLICABILITY

The present invention is useful for determining the nutritional requirements of an industrial animal, and feeds can be formulated, mixed, and provided accordingly.

1. A method for identifying a metabolite highly required by an industrial animal, which comprises:
A) identifying an mRNA, the expression of which changes before and after a change in physiological condition of the industrial animal, comprising comparing expression data of said mRNA obtained before and after the change in physiological condition;
B) identifying a metabolic pathway of which the expression changes before and after the change in the physiological condition on the basis of the identified mRNA; and
C) identifying a metabolite that is highly required by the industrial animal before or after the change in physiological condition on the basis of the identified metabolic pathway.

2. The method according to claim 1, which comprises obtaining mRNA expression data.

3. The method according to claim 1, wherein when the expression of a certain metabolic pathway increases before or after the change in the physiological condition, a metabolite corresponding to upstream of the metabolic pathway and/or a metabolite corresponding to downstream of the metabolic pathway is identified as a metabolite highly required by the industrial animal before or after the change in the physiological condition.

4. The method according to claim 1, wherein when the expression of a certain metabolic pathway decreases before or after the change in the physiological condition, a metabolite corresponding to downstream of the metabolic pathway and important for growth or life activity, and/or a metabolite corresponding to upstream of the metabolic pathway is identified as a metabolite highly required by the industrial animal before or after the change in the physiological condition.

5. The method according to claim 1, wherein said change in physiological condition is selected from group consisting of ontogenesis, a transition in a baby animal from the fetal period to the period after birth, a transition in a mother animal from the gestation period to the non-gestation period, start of feeding of a baby animal from a source other than the mother, a transition in a mother animal from a non-lactation period and a lactation period, and a transition in a baby animal from a lactation period to a non-lactation period.

6. A method for producing a feed composition, which comprises:
identifying a metabolite that is highly required by an industrial animal before or after a change in physiological condition by the method according to claim 1; and
adding the metabolite to a feed composition.
